(19) United States
(12) Patent Application Publication
(10) Pub. No.: US 2024/0261019 A1
(43) Pub. Date: Aug. 8, 2024

(54) OPTIMIZED ELECTRODE SPACING FOR BIPOLAR SPHINCTEROTOMES

(71) Applicant: Cook Medical Technologies LLC, Bloomington, IN (US)

(72) Inventors: Marc Spencer, Winston-Salem, NC (US); Shaun Gittard, Winston-Salem, NC (US); John Crowder Sigmon, JR., Winston-Salem, NC (US); Christopher A. Carruthers, Winston-Salem, NC (US)

(21) Appl. No.: 18/640,750

(22) Filed: Apr. 19, 2024

Related U.S. Application Data
(63) Continuation of application No. PCT/US2022/047000, filed on Oct. 18, 2022.
(60) Provisional application No. 63/257,329, filed on Oct. 19, 2021.

(51) Int. Cl. A61B 18/14; A61B 18/00; A61B 18/12; A61B 18/16

(52) U.S. Cl. CPC A61B 18/1492; A61B 18/1206; A61B 18/16; A61B 2018/00083; A61B 2018/00178; A61B 2018/00184; A61B 2018/00482; A61B 2018/00601; A61B 2018/0091; A61B 2018/126; A61B 2018/144; A61B 2018/162

(57) ABSTRACT

A bipolar sphincterotome may include an elongate tubular member longitudinally extending from a proximal portion to a distal portion, an active path comprising a cutting edge exposed outside of the elongate tubular member, and a return electrode disposed over an outer surface of the elongate tubular member. In various embodiments, in an axial cross-section of the bipolar sphincterotome, the return electrode does not intersect a transverse axis. In addition or alternatively, a minimum distance between the cutting edge and the return electrode is at least about 0.050 inches.

### CROSS-REFERENCE TO RELATED APPLICATIONS

This application is a continuation of International Application No. PCT/US2022/047000, filed Oct. 18, 2022, which claims the benefit of priority to U.S. Provisional Application No. 63/257,329, filed Oct. 19, 2021. The contents of International Application No. PCT/US2022/047000 and U.S. Provisional Application No. 63/257,329 are each incorporated herein by reference in their entirety.

### TECHNICAL FIELD

The present invention relates generally to medical devices, and more particularly to bipolar sphincterotomes.

### BACKGROUND

A sphincterotome is a medical device that is used to perform a sphincterotomy, which involves cutting a sphincter muscle, such as the sphincter of Oddi. The sphincter muscle may need to be cut to relieve its constrictive nature and allow one or more medical devices through the muscle. For example, problems occurring in the biliary tree, such as the formation of bile duct stones or papillary stenosis, may be treated using medical devices that are delivered into the biliary tree. In order to access the biliary tree, the medical devices may pass through the sphincter of Oddi. To facilitate passage of the medical devices through the sphincter of Oddi, the sphincter muscle may be cut using a sphincterotome.

### BRIEF SUMMARY

The present describes various embodiments of a bipolar sphincterotome, electrosurgical systems, and electrosurgical medical devices that have optimized spacing between a cutting edge and a return electrode for minimizing the likelihood of arcing during performance of a sphincterotomy, including those performed within a gastrointestinal tract of a patient and/or to cut a sphincter muscle. In one embodiment, a bipolar sphincterotome includes: an elongate tubular member longitudinally extending from a proximal portion to a distal portion; an active path comprising a cutting edge exposed outside of the elongate tubular member; and a return electrode disposed over an outer surface of the elongate tubular member, wherein in an axial cross-section of the bipolar sphincterotome, the return electrode does not intersect a transverse axis of the bipolar sphincterotome, the transverse axis perpendicular to a longitudinal axis of the bipolar sphincterotome and to a radial direction defined by the cutting edge.

In another embodiment, a bipolar sphincterotome includes: an elongate tubular member longitudinally extending from a proximal portion to a distal portion; an active path comprising a cutting edge disposed outside of the elongate tubular member; and a return electrode disposed over an outer surface of the elongate tubular member, wherein a minimum distance between the cutting edge and the return electrode is at least about 0.050 inches.

Other embodiments are possible, and each of the embodiments can be used alone or together in combination. Accordingly, various embodiments are described below with reference to the accompanying drawings.

### BRIEF DESCRIPTION OF THE DRAWINGS

FIG. 1 shows a cross-sectional side view of an electrosurgical system including a bipolar sphincterotome electrically coupled to a power source.

FIG. 2 shows a perspective view of a distal portion of the bipolar sphincterotome of FIG. 1 in a curled position.

FIG. 3 shows a cross-section axial view of an example configuration of the distal portion of the bipolar sphincterotome of FIG. 1, taken along line 3-3 in FIG. 1.

FIG. 4 shows a cross-sectional axial view of another example configuration of the distal portion of the bipolar sphincterotome of FIG. 1, taken along line 3-3 in FIG. 1.

### DETAILED DESCRIPTION

The present description describes various embodiments of a sphincterotome having a bipolar configuration, otherwise referred to as a bipolar sphincterotome. The present description also describes various embodiments of an electrosurgical system including a bipolar sphincterotome coupled to a power source. In various embodiments, the bipolar sphincterotome has a return electrode disposed over an outer surface of an elongate tubular member that circumferentially does not intersect a transverse axis of the bipolar sphincterotome. In addition or alternatively, the bipolar sphincterotome has a minimum distance between the cutting edge and the return electrode that is at least about 0.050 inches. Such configurations may provide optimized features for a bipolar sphicterotome that sufficiently reduce or minimize the likelihood of arcing between a cutting edge and the return electrode.

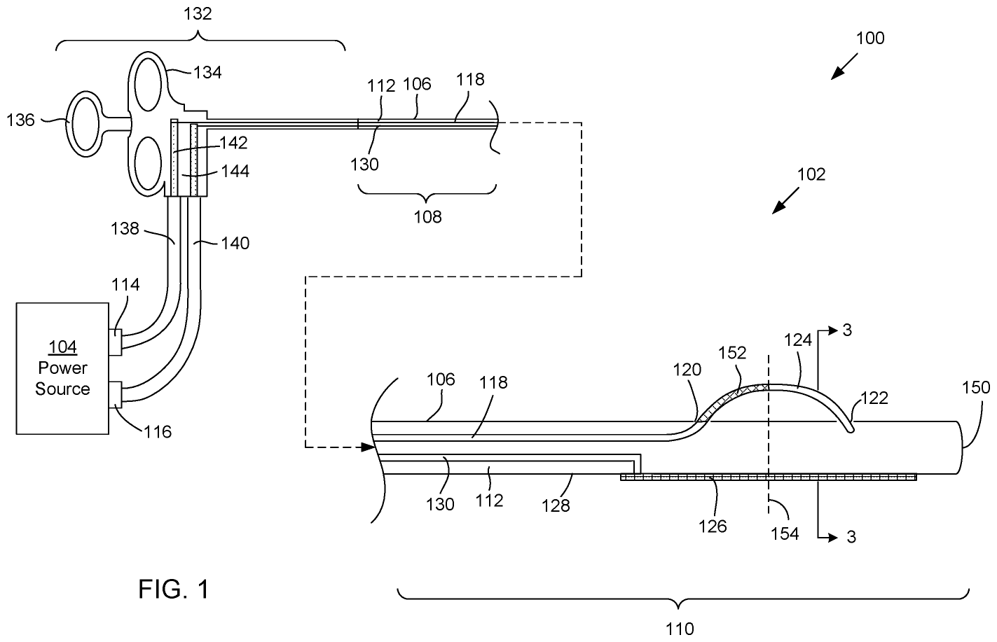

FIG. 1 shows a partial cross-sectional side view of an example electrosurgical system 100 that includes a bipolar sphincterotome 102 coupled a power source 104. Other embodiments of the present description may include only the bipolar sphincterotome 102 without the power source 104.

The bipolar sphincterotome 102 may include an elongate tubular member 106 (e.g., a catheter) that longitudinally extends from a proximal portion 108 to a distal portion 110. The elongate tubular member 106 may include a body 112 extending from the proximal portion 108 to the distal portion 110. The body 112 may be made of various suitable materials, including non-conductive materials, such as polytetrafluoroethylene (PTFE), perfluoroalkoxy (PFA), polyethylene, nylon, or fluorinated ethylene, as non-limiting examples. In addition, in various embodiments, the body 112 of the elongate tubular member 106 may have an outer diameter in a range of about 0.050 inches to about 0.100 inches. In particular embodiments, the outer diameter is 0.085 inches.

The bipolar sphincterotome 102 may further include an active path and a return path. In general, the active path and the return path are each conductive elements, or each a network of elements, that are configured to deliver electrical current between the power source 104 and a treatment site (e.g., a sphincter muscle) within a patient. As shown in FIG. 1, the power source 104 (e.g, an electrosurgical unit (ESU) or a radio frequency (RF) generator) may be configured to output electrical current via a pair of ports, including an active port 114 and a return port 116. The active path may be configured to electrically couple to the active port 114, and the return path may be configured to electrically couple to the return port 116. In addition, at a treatment site within a patient, a distal portion of the active path is configured to contact one portion of tissue at the treatment site, and a distal portion of the return path is configured to contact another portion of the tissue. The tissue itself provides a path of relatively low resistance.

Accordingly, when the distal portions of the active and return paths are contacting the tissue, the power source, the active and return paths, and the tissue at the treatment site form an electrical circuit through which electrical current generated by the power source 104 may flow. The electrical current flowing through the tissue may produce a certain effect on the tissue, such as cutting, ablation, or coagulation, as non-limiting examples. In addition, for an electrosurgical procedure such as a sphincterotomy, the electrical current may be an alternating current (AC), such as a radio frequency (RF) current. Accordingly, when the electrical circuit is formed, depending on its polarity, electrical current generated by the power source 104 may flow from the active port 114 of the power source 104, through the active path of the bipolar sphincterotome 102, through the tissue at the treatment site, and then through the return path back to the return port 116; or may flow from the return port 116, through the return path, through the tissue at the treatment site, and then through the active path back to the active port 114.

For the bipolar sphincterotome in FIG. 1, the active path may include a conductive, active wire 118 (also called a cutting wire) that longitudinally extends from the proximal portion 108 to the distal portion 110. In various embodiments, such as shown in FIG. 1, the active wire 118 may longitudinally extend within the body 112 except at the distal portion 110, where the active wire 118 exits at an exit port 120 from within the body 112 to outside the body 112, and distally extends outside the body 112 from the exit port 120 to an anchor point 122, where a distal end of the active wire 118 is secured or affixed to the body 112. The portion of the active wire 118 that is outside of the body 112 may be referred to as a cutting edge 124, and is an electrode part of the active wire 118 that is configured to contact tissue at the treatment site. For example, during a sphincterotomy, the cutting edge 124 is a part of the active wire 118 that is configured to contact a sphincter muscle and cut the sphincter muscle when electrical current is delivered to it. In various embodiments, the cutting edge 124 may have a diameter in a range of about 0.010 inches to about 0.024 inches. Additionally, in various embodiments, a remaining portion of the active wire 118 other than the cutting edge 124 may have the same diameter or a different diameter than the cutting edge 124.

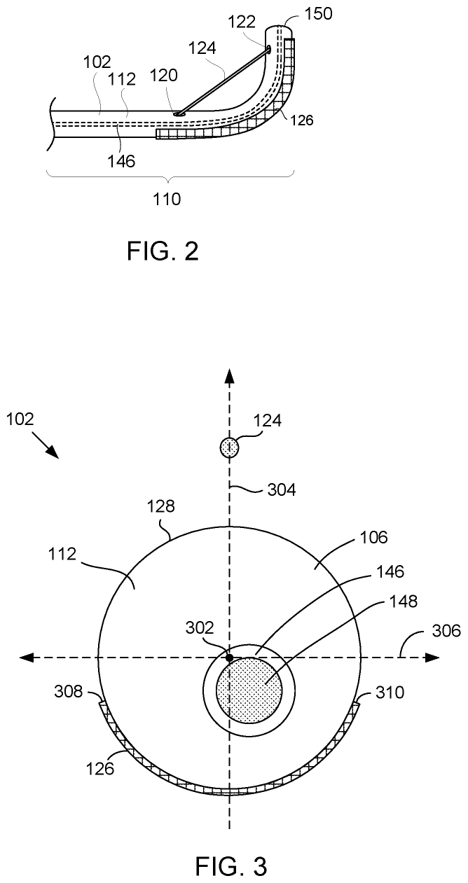

In addition, the active wire 118 may be longitudinally movable within the body 112, such as by being disposed within an active wire lumen (not shown in FIG. 1) that longitudinally extends from the proximal portion 108 to the distal portion 110. By being longitudinally movable within the body 112, the active wire 118 may be configured to move the distal portion 110 of the elongate tubular member 106 between curled and uncurled positions. FIG. 1 shows the distal portion 110 in an uncurled position. FIG. 2 shows the distal portion 110 in a curled position. For example, when the active wire 118 is proximally pulled relative to the elongate tubular member 106, the distal end of the active wire 118 may exert a force on the elongate tubular member 106 at the anchor point 122 that causes the distal portion 110 to curl in a curling direction, as shown in FIG. 2. When distal portion 110 is in a curled position due to the active wire 118 being proximally pulled, the cutting edge 124 may be relatively taut and in a cutting position. An operator operating the bipolar sphincterotome 102 to perform a sphincterotomy may operate the bipolar sphincterotome 102 to proximally pull the active wire 118 in order to configure the distal portion 110 in the curled position and the cutting edge 124 to be taut in the cutting position in order to cut the tissue.

Additionally, the return path of the bipolar sphincterotome 102 may include a return electrode 126 at the distal portion 110. In general, the return electrode 126 is a conductive component of the return path that is exposed from within the elongate tubular member 106 and is configured to contact tissue at the treatment site. Additionally, in various embodiments, such as shown in FIG. 1, the return electrode 126 may cover, be disposed over or about, and/or affixed or adhered to an outer surface 128 of the body 112 of the elongate tubular member 106. The return electrode 126 may be made of any various conductive materials, such as conductive ink, paste, foil, wires, or sheet metal, as non-limiting examples. Also, although not expressly shown in the figures, the bipolar sphincterotome may include one or more intermediate layers, such as an epoxy layer, that adheres the conductive material of the return electrode 126 to the outer surface 128 of the body 112. In this context, the return electrode 126 covering, being disposed over or about, and/or affixed or adhered to the outer surface 128, does not necessarily require that the conductive material of the return electrode 126 is in direct contact with the outer surface 128 of the body 112. Additionally, in various embodiments, the return electrode 126 may extend a longitudinal length over which the cutting edge 124 also longitudinally extends, from the exit port 120 to the anchor point 122. In various embodiments, the cutting edge 124 has a longitudinal length of 7 millimeters, although other lengths may be possible. Also, for at least some of these embodiments, the return electrode 126 may further longitudinally extend at least one of: proximally past the exit port 120 of the elongate tubular member 106 or distally past the anchor point 122. FIGS. 1 and 2 show the return electrode 126 both proximally extending past the exit port 120 and distally extending past the anchor point 122.

Additionally, the return path may include a return wire or other elongate conductive element 130 that longitudinally extends from the proximal portion 108 to the distal portion 110 and that connects to the return electrode 126. In various embodiments, such as shown in FIG. 1, the return wire 130 may longitudinally extend within the body 112, such as by extending within a return lumen (not shown in FIGS. 1 and 2) of the elongate tubular member 106. In other embodiments, at least a portion of the return wire 130 extends outside, such as alongside, the body 112. Various ways of connecting the return wire 130 to the return electrode 126 and/or having the return path longitudinally extend from the proximal portion 108 to the distal portion 110 may be possible.

The bipolar sphincterotome 102 may further include a handle assembly 132 operably coupled to the elongate tubular member 106 and the active wire 118. In various embodiments, the handle assembly 132 may include a plurality of portions that are movable relative to each other to control longitudinal movement of the active wire 118 relative to the elongate tubular member 106. For example, in the example configuration shown in FIG. 1, the handle assembly 132 may include a first portion 134 in the form of finger rings and a second portion 136 in the form of a thumb ring through which an operator may insert his/her fingers and thumb, respectively, when grasping the handle assembly 132. The thumb ring 136 may be operatively coupled to the active wire 118, and the finger rings 134 may be operatively coupled to the elongate tubular member 106, such that movement of the thumb ring 136 relative to the finger rings 134 may cause the active wire 118 to longitudinally move relative to the elongate tubular member 106. Correspondingly, upon grasping the handle assembly 132, the operator may open and close his/her hand to move the thumb ring 136 farther away and closer to the finger rings 134, respectively. Moving the finger rings 134 toward the thumb ring 136 may proximally pull the active wire 118, which in turn may move the distal portion 110 into the curled position, such as shown in FIG. 2. Additionally, moving the finger rings 134 away from the thumb ring 136 may move the distal portion 110 into the uncurled position, such as shown in FIG. 1. The handle assembly 132 shown in FIG. 1 is merely exemplary, and various other configurations of a handle assembly to facilitate longitudinal movement of the active wire 118 relative to the elongate tubular member 106 may be possible.

Additionally, in various embodiments, the electrosurgical system 100 may include electrical cabling configured electrically couple the power source 104 to the bipolar sphincterotome 102. The electrical cabling may include an active cable 138 configured to connect to the active port 114 and a return cable 140 configure to connect to the return port 116. In various embodiments, the electrical cabling may be considered a component of the bipolar sphincterotome 102. In other embodiments, the electrical cabling may be considered a component of the electrosurgical system 100 that is separate from the bipolar sphincterotome 102.

Also, in various embodiments such as shown in FIG. 1, the handle assembly 132 may include components of one or both of the active path or the return path. For example, the active cable 138 may be configured to connect to the handle assembly 132, and a conductive active element 142 integrated with the handle assembly 132 may electrically connect the active cable 138 to the active wire 118 when the active cable 138 is connected to the handle assembly 132. In addition or alternatively, the return cable 140 may be configured to connect to the handle assembly 132, and a conductive return element 144 integrated with the handle assembly 132 may electrically connect the return cable 140 to the return wire 130 when the return cable 140 is connected to the handle assembly 132. In various embodiments, the active cable 138 and/or the return cable 140 may be fixedly connected or attached to the handle assembly 132. In other embodiments, the active cable 138 and/or the return cable 140 may be removably connected or attached to the handle assembly 132.

FIG. 3 shows an axial cross-sectional view of the bipolar sphincterotome 102 taken along line 3-3 in FIG. 1. The axial cross-sectional view in FIG. 3 is generally representative of an axial cross-section of the bipolar sphincterotome 102 at the distal portion 110 where the cutting edge 124 outside of the body 112 of the elongate tubular member 106 is disposed. Referring also to FIG. 2, the bipolar sphincterotome 102 may include a wire guide lumen 146 through which a wire guide 148 may be movably disposed. During an operation, the wire guide 148 may be inserted into the patient and a distal end of the wire guide 148 may be moved to the treatment site. The wire guide lumen 146 may then be inserted over the wire guide 148, and the distal portion 110 of the bipolar sphincterotome 102 may be distally advanced to the treatment site within the patient. In this way, the wire guide 148 facilitates delivery of the distal portion 110 of the bipolar sphincterotome 102 to the treatment site. Generally, the wire guide lumen 146 longitudinally extends within the body 112 from the proximal portion 108 to a distal end 150, such as shown in FIG. 2. Additionally, for at least some embodiments, one or more lumens for the active wire 118 and/or the return wire 130 may stop or terminate at or proximal to the exit port 120, such that only one lumen, such as the wire guide lumen 146, longitudinally extends through the body 112 over the longitudinal length that the cutting edge 124 longitudinally extends, as depicted in FIG. 3. Other configurations, including those where more than one lumen longitudinally extends through the body 112 over the longitudinal length that the cutting edge 124 longitudinally extends, may be possible.

Referring particularly to FIG. 3, geometrically, the elongate tubular member 106 may be centrally longitudinally positioned about a central longitudinal axis, which is represented by a centrally-positioned point 302 in FIG. 3. Further, within an axial cross-section, a radial direction 304 defined by the cutting edge 124 extends from the longitudinal axis (e.g., from point 302) through the cutting edge 124. Additionally, a transverse axis 306 intersects the longitudinal axis 302 and is perpendicular to both the longitudinal axis 302 and the radial direction 304.

Additionally, as shown in FIG. 3, in an axial cross-section, the return electrode 126 may circumferentially extend from a first end 308 to a second end 310 about the outer surface 128. When designing a bipolar sphincterotome, one of ordinary skill in the art may intuitively configure the return electrode 126 to circumferentially extend at least 180 degrees, or at least half a circumference, about the outer surface 128 of the elongate tubular member 106. In doing so, in an axial cross-section, the return electrode 126 would geometrically intersect the transverse axis 308 at least once (in at least one location). For example, a return electrode 126 circumferentially extending at least 180 degrees may have its ends 308, 310 disposed on or overlapping the transverse axis 306, or the return electrode 126 may intersect or “crossover” the transverse axis 306 at least once as it circumferentially extends to its ends 308, 310. A designer may intuitively configure the return electrode 126 to circumferentially extend at least 180 degrees to ensure that the return electrode 126 has a sufficient amount of surface area to contact tissue at the treatment site for a bipolar configuration, and at the same time, the return electrode 126 would not be too close to the cutting edge 124 to create undesirable arcing.

However, during testing, the inventors discovered that a minimum distance between the cutting edge 124 and the return electrode 126 should be at least about 0.050 inches to sufficiently protect against arcing between the cutting edge 124 and the return electrode 126. Herein, the minimum distance between the cutting edge 124 and the return electrode 126 is a shortest point-to-point distance of all possible paths extending from any point on the cutting edge 124 to any point on the return electrode 126 that do not extend through the bipolar sphincterotome 102.

During performance of a sphincterotomy, such as within the gastrointestinal (GI) tract, the treatment site contains a mixture of tissue, body fluid such a bile, and air or other gases. Heat generated from delivery of electrical current to the treatment site may further affect the composition of the mixture at the treatment site. During development of the bipolar sphincterotome embodiments described herein, the inventors created a test setup that simulated the environment of a treatment site within a patient where a sphincterotomy is performed. The test setup included an active wire with a cutting edge and a return electrode separated by bile, similar to the actual environment in the patient's GI tract. The active wire and return electrode were electrically coupled to a power generator, which delivered electrical current to the electrodes at power settings matching those used during a sphincterotomy. Distances between the cutting edge and return electrode were varied while subjected to the bile and electrical current was being delivered.

Through testing, the inventors discovered that a minimum distance smaller than about 0.050 inches provides too high of a likelihood of arcing between the cutting edge 124 and the return electrode 126 over a path containing some combination of the mixture. Accordingly, in various embodiments described herein, including those where the outer diameter of the elongate tubular member 106 is in a range of 0.050 inches to 0.100 inches, the bipolar sphincterotome 102 has a minimum distance between the cutting edge 124 and the return electrode 126 of at least 0.050 inches. In some of these embodiments, an upper bound of the minimum distance is dependent on or restricted by a width (i.e., a circumferential distance) of the return electrode 126. In particular embodiments, the width of the return electrode is at least (greater than or equal to) 0.030 inches. Correspondingly, the minimum distance between the cutting edge 124 and the return electrode 126 may be any value greater than or equal to 0.050 inches, further provided, or on condition, that the width of the return electrode 126 is at least 0.030 inches. Configuring the return electrode 126 to have a width of at least 0.030 inches may ensure that the return electrode 126 is not too small or thin so that the return electrode 126 and the cutting edge 124 have a surface area ratio of at least 3:1, and/or so that the width is sufficiently large so as to ensure sufficient contact with tissue during operation. In addition or alternatively, in some of these embodiments, the minimum distance is in a range of 0.060 inches to 0.080 inches. In particular of these embodiments, the outer diameter of the elongate tubular member 106 is 0.085 inches, and the minimum distance is at least 0.062 inches.

Correspondingly, in some embodiments, for an elongate tubular member 106 with an outer diameter in a range of about 0.050 inches to about 0.100 inches to have the minimum distance greater than or equal to 0.050 inches, the return electrode 126 circumferentially extends over the outer surface 128 so as not to intersect the transverse axis 306, resulting in an unexpected configuration of the return electrode 126. The return electrode 126 not intersecting the transverse axis 306 is shown in FIG. 3. For example, the ends 308, 310 are each disposed “under” or “below” the transverse axis 306, with the return electrode 126 circumferentially extending less than 180 degrees about the outer surface 128 of the elongate tubular member 106.

Referring back to FIG. 1, for at least some embodiments, a portion of the cutting edge 124 may not be configured to contact and cut tissue, despite being disposed outside of the tubular member 106. For example, as shown in FIG. 1, an insulating cover, such as a tube or sleeve, 152 may cover or be disposed about a portion of the cutting edge 124 such that it prevents that portion of the cutting edge 124 from contacting tissue and delivering electrical current to the tissue. In other words, the insulating cover 152, rather than the portion of the cutting edge 124 it is covering, is configured to contact tissue. The insulating cover 152 is made of an insulating material (such as polytetrafluoroethylene (PTFE) or polyimide, although other insulating materials are possible), and is therefore configured to not deliver electrical current to the tissue it is contacting. Including the insulating cover 152 may reduce the surface area of the cutting edge 124 configured to contact tissue, compared to if no insulating cover 152 was present, which in turn may improve the ability of the cutting edge 124 to cut the tissue.

For at least some embodiments such as shown in FIG. 1, the insulating cover 152 extends distally from the exit port 120. The insulating cover 152 may extend about half a longitudinal length of the cutting edge 124, although other longitudinal lengths of the insulating cover 152 may be possible.

A distal end or termination of the insulating cover 152, and/or a proximal end of the cutting edge 124 that is uninsulated and configured to contact tissue, may define a plane 154 perpendicular to the longitudinal axis of the elongate tubular member 106. The plane is represented by dotted line 154 in FIG. 1. For at least some embodiments, over a longitudinal length of the bipolar sphincterotome 102 extending distally from the plane 154 to the distal end 150, a ratio of the surface area of the return electrode 126 to the surface area of the cutting edge 124 may be at least about three-to-one (3:1). Such a surface area ratio between the cutting edge 124 and the return electrode 126 may provide a sufficient or optimal contact ratio and/or current density ratio between the cutting edge 124 and the return electrode 126 for performance of a sphincterotomy. Correspondingly, the return electrode 126 may be sized so that a minimum distance between the cutting edge 124 and the return electrode 126 is at least about 0.050 inches, and a surface area ratio between the return electrode 126 and the cutting edge 124 over a longitudinal length extending distally from the perpendicular plane 154 defined by the uninsulated proximal end of the cutting edge 124 is at least 3:1.

Referring back to FIG. 3, the axial cross-section in FIG. 3 may be representative of axial cross-sections of the bipolar sphincterotome 102 over at least half a longitudinal length of the return electrode 126. That is, the return electrode 126 may not intersect the transverse axis 306 in axial cross-sections longitudinally extending over at least half a longitudinal length of the return electrode 126. In particular embodiments, the return electrode 126 may not intersect the transverse axis 306 in axial cross-sections longitudinally extending over its entire longitudinal length. For at least some embodiments, the entire or greatest longitudinal length of the return electrode 126 is about five centimeters (cm), although other lengths may be possible.

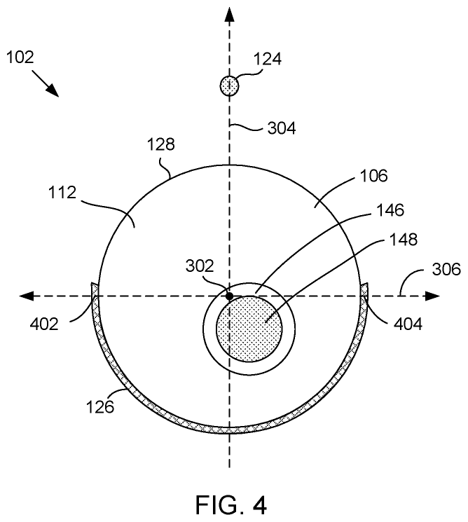

In various other embodiments described herein, a bipolar sphincterotome may have a minimum distance between the cutting edge 124 and the return electrode 126 of at least about 0.050 inches, and the return electrode 126 intersects the transverse axis 306 in at least one location. In some of these embodiments, the return electrode 126 may intersect the transverse axis 306 in at least one location over at least half a longitudinal length of the return electrode 126. In particular of these embodiments, the return electrode 126 may intersect the transverse axis 306 in at least one location over an entirety of the longitudinal length of the return electrode 306. To illustrate, FIG. 4 shows an example configuration the return electrode 126 circumferentially extends more than half of the circumference of the elongate tubular member 106 (e.g., more than 180 degrees), such that the return electrode 126 intersects the transverse axis 306 in two locations 402, 404. In some of these embodiments, the elongate tubular member 106 may have an outer diameter in a range of 0.050 inches to 0.100 inches, while the minimum distance is at least 0.050 inches. In other embodiments, the elongate tubular member 106 may have an outer diameter that is larger than 0.100 inches, such that the return electrode 126 circumferentially extends more than 180 degrees while the minimum distance between the cutting edge 124 and the return electrode 126 is still at least about 0.050 inches. In particular of these embodiments, the minimum distance may be in a range of 0.060 inches to 0.080 inches, as mentioned.

The subject matter of the present description may also relate, among others, to the following aspects:

In a first aspect, a bipolar sphincterotome includes: an elongate tubular member longitudinally extending from a proximal portion to a distal portion; an active path comprising a cutting edge exposed outside of the elongate tubular member; and a return electrode disposed over an outer surface of the elongate tubular member, wherein in an axial cross-section of the bipolar sphincterotome, the return electrode does not intersect a transverse axis of the bipolar sphincterotome, the transverse axis perpendicular to a longitudinal axis of the bipolar sphincterotome and to a radial direction defined by the cutting edge.

In a second aspect, the bipolar sphincterotome of the first aspect further includes that a minimum distance between the cutting edge and the return electrode is at least about 0.050 inches.

In a third aspect, the bipolar sphincterotome of the second aspect further includes that the minimum distance is in a range of 0.060 inches to 0.080 inches.

In a fourth aspect, the bipolar sphincterotome of the first through third aspects further includes that over a longitudinal length distally extending from a plane perpendicular to the longitudinal axis and defined by a proximal end of an uninsulated portion of the cutting edge, a ratio of a surface area of the return electrode to a surface area of the cutting edge is at least about 3:1.

In a fifth aspect, the bipolar sphincterotome of any of the first through fourth aspects further includes that an outer diameter of the elongate tubular member is in a range of about 0.050 inches to about 0.100 inches.

In a sixth aspect, the bipolar sphincterotome of any of the first through fifth aspects further includes that the return electrode longitudinally extends at least one of: proximally past an exit port for the cutting edge or distally past an anchor point for the cutting edge.

In a seventh aspect, the bipolar sphincterotome of the fifth aspect further includes that the return electrode distally extends past the anchor point.

In an eighth aspect, the bipolar sphincterotome of any of the first through seventh aspects further includes that the return electrode longitudinally extends over a distal portion of the elongate tubular member that is configured to curl.

In a ninth aspect, the bipolar sphincterotome of any of the first through eighth aspects further includes that the return electrode does not intersect the transverse axis over at least half a longitudinal length of the return electrode.

In a tenth aspect, the bipolar sphincterotome of the ninth aspect further includes that the return electrodes does not intersect the transverse axis over an entirety of the longitudinal length.

In an eleventh aspect, the bipolar sphincterotome of any of the first through tenth aspects further includes that the elongate tubular member includes at least one lumen in the axial cross-section.

In a twelfth aspect, a bipolar sphincterotome includes: an elongate tubular member longitudinally extending from a proximal portion to a distal portion; an active path comprising a cutting edge disposed outside of the elongate tubular member; and a return electrode disposed over an outer surface of the elongate tubular member, wherein a minimum distance between the cutting edge and the return electrode is at least about 0.050 inches.

In a thirteenth aspect, the bipolar sphincterotome of the twelfth aspect further includes that the minimum distance is in a range of 0.060 inches to 0.080 inches.

In a fourteenth aspect, the bipolar sphincterotome of any of the twelfth or thirteenth aspects further includes that an outer diameter of the elongate tubular member is in a range of about 0.050 inches to about 0.100 inches.

In a fifteenth aspect, the bipolar sphincterotome of any of the twelfth through fourteenth aspects further includes that the return electrode longitudinally extends at least one of: proximally past an exit port for the cutting edge or distally past an anchor point for the cutting edge.

In a sixteenth aspect, the bipolar sphincterotome of the fifteenth aspect further includes that the return electrode distally extends past the anchor point.

In a seventeenth aspect, the bipolar sphincterotome of any of the twelfth through sixteenth aspects further includes that the return electrode longitudinally extends over a distal portion of the elongate tubular member that is configured to curl.

In an eighteenth aspect, the bipolar sphincterotome of any of the twelfth through seventeenth aspects further includes that the return electrode circumferentially extends less than 180 degrees about an outer surface of the elongate tubular member longitudinally over at least half a longitudinal length of the return electrode.

In a nineteenth aspect, the bipolar sphincterotome of the eighteenth aspect further includes that the return electrode circumferentially extends less than 180 degrees about the outer surface over the entirety of the longitudinal length.

In a twentieth aspect, the bipolar sphincterotome of any of the twelfth through seventeenth aspects, and further includes wherein the return electrode circumferentially extends more than 180 degrees about an outer surface of the elongate tubular member such that the return electrode intersects a transverse axis of the bipolar sphincterotome in at least one location.

In a twenty-first aspect, the bipolar sphincterotome of any of the eleventh through twentieth aspects further includes that over a longitudinal length distally extending from a plane perpendicular to a longitudinal axis of the bipolar sphincterotome and defined by a proximal end of an uninsulated portion of the cutting edge, a ratio of a surface area of the return electrode to a surface area of the cutting edge is at least about 3:1.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A bipolar sphincterotome comprising:
an elongate tubular member longitudinally extending from a proximal portion to a distal portion;
an active path comprising a cutting edge exposed outside of the elongate tubular member; and
a return electrode disposed over an outer surface of the elongate tubular member, wherein in an axial cross-section of the bipolar sphincterotome, the return electrode does not intersect a transverse axis of the bipolar sphincterotome, the transverse axis perpendicular to a longitudinal axis of the bipolar sphincterotome and to a radial direction defined by the cutting edge,
wherein a minimum distance between the cutting edge and the return electrode is at least about 0.050 inches.

2. The bipolar sphincterotome of claim 1, wherein a minimum distance between the cutting edge and the return electrode is in a range of 0.060 inches to 0.080 inches.

3. The bipolar sphincterotome of claim 1, wherein, over a longitudinal length distally extending from a plane perpendicular to the longitudinal axis and defined by a proximal end of an uninsulated portion of the cutting edge, a ratio of a surface area of the return electrode to a surface area of the cutting edge is at least about 3:1.

4. The bipolar sphincterotome of claim 1, wherein an outer diameter of the elongate tubular member is in a range of about 0.050 inches to about 0.100 inches.

5. The bipolar sphincterotome of claim 1, wherein the return electrode longitudinally extends at least one of: proximally past an exit port for the cutting edge or distally past an anchor point for the cutting edge.

6. The bipolar sphincterotome of claim 5, wherein the return electrode distally extends past the anchor point.

7. The bipolar sphincterotome of claim 1, wherein the return electrode longitudinally extends over a distal portion of the elongate tubular member that is configured to curl.

8. The bipolar sphincterotome of claim 1, wherein the return electrode does not intersect the transverse axis over at least half a longitudinal length of the return electrode.

9. The bipolar sphincterotome of claim 8, wherein the return electrode does not intersect the transverse axis over an entirety of the longitudinal length.

10. The bipolar sphincterotome of claim 1, wherein the elongate tubular member comprises at least one lumen in the axial cross-section.

11. A bipolar sphincterotome comprising:
an elongate tubular member longitudinally extending from a proximal portion to a distal portion;
an active path comprising a cutting edge disposed outside of the elongate tubular member; and
a return electrode disposed over an outer surface of the elongate tubular member,
wherein a minimum distance between the cutting edge and the return electrode is at least about 0.050 inches.

12. The bipolar sphincterotome of claim 11, wherein the minimum distance is in a range of 0.060 inches to 0.080 inches.

13. The bipolar sphincterotome of claim 11, wherein an outer diameter of the elongate tubular member is in a range of about 0.050 inches to about 0.100 inches.

14. The bipolar sphincterotome of claim 11, wherein the return electrode longitudinally extends at least one of: proximally past an exit port for the cutting edge or distally past an anchor point for the cutting edge.

15. The bipolar sphincterotome of claim 14, wherein the return electrode distally extends past the anchor point.

16. The bipolar sphincterotome of 11, wherein the return electrode longitudinally extends over a distal portion of the elongate tubular member that is configured to curl.

17. The bipolar sphincterotome of claim 11, wherein the return electrode circumferentially extends less than 180 degrees about an outer surface of the elongate tubular member longitudinally over at least half a longitudinal length of the return electrode.

18. The bipolar sphincterotome of claim 17, wherein the return electrode circumferentially extends less than 180 degrees about the outer surface over the entirety of the longitudinal length.

19. The bipolar sphincterotome of claim 11, wherein the return electrode circumferentially extends more than 180 degrees about an outer surface of the elongate tubular member such that the return electrode intersects a transverse axis of the bipolar sphincterotome in at least one location.

20. The bipolar sphincterotome of claim 11, wherein, over a longitudinal length distally extending from a plane perpendicular to a longitudinal axis of the bipolar sphincterotome and defined by a proximal end of an uninsulated portion of the cutting edge, a ratio of a surface area of the return electrode to a surface area of the cutting edge is at least about 3:1.
